(19) United States
(12) Patent Application Publication
(10) Pub. No.: US 2005/0187304 A1
(43) Pub. Date: Aug. 25, 2005

(54) Methods and compositions comprising non-peptide small molecules that solubilize the alzheimer's Abeta peptide fiber

(76) Inventors: Yves Claude Nicolau, Newton, MA (US); Alexey Eliseev, Brookline, MA (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/783,699

(22) Filed: Feb. 20, 2004

(51) Int. Cl. A61K 31/015

(52) U.S. Cl. 514/764

(57) ABSTRACT

The present invention comprises novel compositions and methods for the treatment of Alzheimer's disease. In particular, the present invention is directed to Aβ fiber solubilization, for the treatment of Alzheimer's disease, and is described herein as being achieved by using small molecules of non-peptide nature.

### FIELD OF THE INVENTION

The present invention is related to methods and compositions for treating Alzheimer's disease. In particular, the present invention comprises novel compositions and methods for destroying the formation of plaques formed via aggregation of fibers of amyloid peptide in its beta sheet conformation (Aβ).

### BACKGROUND OF THE INVENTION

Alzheimer's disease (AD) is a progressive disease of the brain that is characterized by impairment of memory and a disturbance in at least one other thinking function (for example, language or perception of reality). Scientific evidence demonstrates that AD results from an increase in the production or accumulation of a specific protein (beta-amyloid protein) that leads to nerve cell death. Loss of nerve cells in strategic brain areas, in turn, causes deficits in the neurotransmitters, which are the brain's chemical messengers.

Alzheimer's disease is not a normal part of aging and is not something that inevitably happens in later life. Rather, it is one of the dementing disorders, which are a group of brain diseases that result in the loss of mental and physical functions.

The main risk factor for AD is increased age. As the population ages, the frequency of AD continues to increase. 10% of people over age 65 and 50% of those over 85 have AD. The number of individuals with AD is expected to be 14 million by the year 2050. In 1998, the annual cost for the care of patients with AD in the United States was approximately $40,000 per patient.

There are also genetic risk factors for AD. The presence of several family members with AD has suggested that, in some cases, heredity may influence the development of AD. A genetic basis has been identified through the discovery of mutations in several genes that cause AD in a small subgroup of families in which the disease has frequently occurred at relatively early ages (beginning before age 50). Some evidence points to chromosome 19 as implicated in certain other families in which the disease has frequently developed at later ages.

Studies of aging and dementia (general mental deterioration) in the general population have identified three groups of subjects; persons who are not demented, those who are demented, and individuals who cannot be classified because they have a cognitive (thinking, memory) impairment, but do not meet the criteria for dementia.

With the exception of rare cases of familial AD, in whom the disease is caused by mutations (changes in the DNA) of a single gene, most cases of AD are probably caused by a variety of factors acting together. Cases without a family history are called “sporadic.” The study of familial AD, however, has uncovered several proteins that are not only important for familial, but also for sporadic AD. These are the amyloid precursor protein (APP) and two presenilins. APP is a major component of plaques (abnormal deposits of proteins in the brain).

The break-down (faulty cleavage) of APPs likely increases their propensity to combine (aggregate) in plaques. Presenilins, on the other hand, are involved in the splitting (cleavage) of APP. Mutations in the genes that encode APPs and the presenilins can cause AD. This means that individuals carrying these mutations have a very high probability of developing AD.

The management of AD consists of medication-based and non-medication based treatments. Treatments aimed at changing the underlying course of the disease (delaying or reversing the progression) have so far been largely unsuccessful. Medicines that restore the deficit (defect), or malfunctioning, in the chemical messengers of the nerve cells (neurotransmitters), such as the cholinesterase inhibitors, have been shown to improve symptoms. Finally, medications are available that deal with the psychiatric manifestations of AD.

ChEIs are the only agents that are approved by the FDA for the treatment of AD. ChEIs are medicines that restore the defect, or malfunctioning, in the chemical messengers of the nerve cells. These chemical messengers are referred to as neurotransmitters. ChEIs impede the action of an enzyme that inactivates the chemical messengers. Therefore, in the presence of ChEI medicines, more chemical messengers are available to transmit the messages of the nerves in the brain. Although significant treatment effects have been demonstrated indicating that this class of agents is consistently better than a placebo, the disease continues to progress despite treatment, and the average effect on mental functioning has only been modest.

In addition, ChEIs have effects on many aspects of daily living. The principal side effects of ChEIs involve the gastrointestinal system and include nausea, vomiting, cramping, and diarrhea. Tacrine (one of the approved ChEIs) is the only agent that is associated with liver toxicity and the use of this agent requires close hematologic (blood test) monitoring, including liver function tests every other week during the period of dose escalation and every 3 months thereafter. Rivastigmine (another of the approved ChEIs) has been associated with weight loss and the monitoring of the patient's weight is recommended when using this drug.

The occurrence of side effects for ChEIs is related to the rate of dose increase. Therefore, doctors gradually increase the dose at intervals until the optimal therapeutic dosage has been reached. The interval between dose increases may be extended or the dose step size may be reduced accordingly if side effects occur. Between 75% and 90% of patients will tolerate therapeutic doses of ChEIs.

Advances in the understanding of the brain abnormalities that occur in AD are hoped to provide the framework for new targets of treatment that are more focused on altering the course and developmentof the disease. Many compounds, including anti-inflammatory agents, are being actively investigated. Clinical trials using specific cyclooxygenase inhibitors (COX-2), such as rofecoxib and celecoxib, are underway.

What is needed therefore, are effective compositions and methods for addressing the complications associated with Alzheimer's disease. In particular what is need are novel pharmaceuticals capable of counteracting the physiological manifestations of the disease such as the formation plaques associated with aggregation of fibers of the amyloid peptide in its beta sheet conformation (Aβ).

### SUMMARY OF THE INVENTION

The present invention is directed to novel compositions and methods for the treatment of Alzheimer's disease. In particular, the present invention is directed to Aβ fiber solubilization, which is considered to be an approach for the treatment of Alzheimer's disease, and is described herein as being achieved by using small molecules of non-peptide nature. Several classes of compounds are described herein that, according to computer-aided design, are able to provide such a solubilization effect. Also described are methods for designing and testing such molecules for their in vitro and in vivo activity.

Accordingly, an object of the present invention is to provide novel compositions and methods for the treatment of Alzheimer's disease.

Another object of the present invention is to provide novel compositions and methods for the treatment of Alzheimer's disease comprising small non-peptide molecules that solubilize Alzheimer's Aβ peptide fiber.

Another object of the present invention is to provide methods and compositions for reducing and preventing the formation plaques associated with associated with aggregation of fibers of the amyloid peptide in its beta sheet conformation (Aβ).

Yet another object of the present invention is to provide the novel compositions that may be administered intramuscularly, intravenously, transdermally, orally, or subcutaneously.

These and other objects, features and advantages of the present invention will become apparent after a review of the following detailed description of the disclosed embodiment and the appended claims.

### BRIEF DESCRIPTION OF THE FIGURES

FIG. 1 provides a schematic showing a chemical approach to combinatorial discovery of P-sheet breaking small molecules.

FIG. 2 provides a schematic showing the “morphomer” concept.

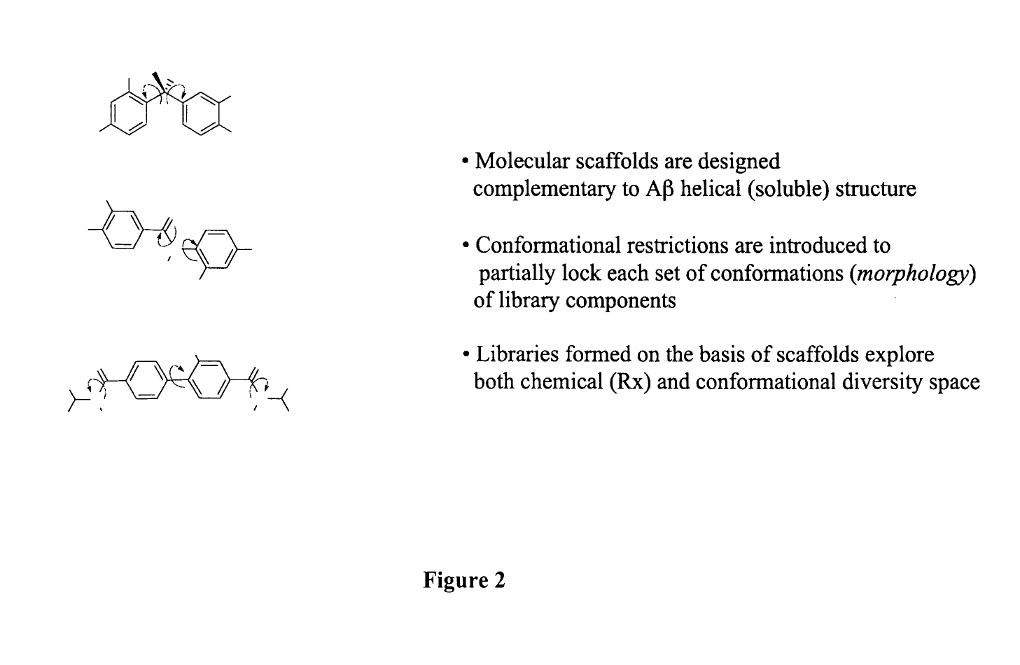

FIG. 3 provides a schematic showing molecular adaptation of morphomers to the target leading to the formation of strong complexes.

FIG. 4 provides a general strategy of small molecules-β-sheet breakers for therapy of Alzheimer's disease.

FIG. 5 provides a schematic for screening of small molecules for Aβ solubilization activity.

### DETAILED DESCRIPTION

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

According to the current understanding, development of the Alzheimer's disease is directly related to the formation of plaques formed via aggregation of fibers of the amyloid peptide in its beta-sheet conformation (Aβ). Therefore, any agent capable of directly destroying the plaques, either via solubilization of Aβ or via its chemical decomposition, is potentially interesting for the Alzheimer's disease therapy.

The β-sheet fiber form of Aβ is formed through a slow conformational change from its soluble form, which is a combination of the α-helical and random coil structures, followed by aggregation. ² Thus, one possible mechanism for solubilization of plaques is via thermodynamic stabilization of the soluble form and thereby reversing the fiber precipitation equilibrium. This concept, referred to as β-sheet breaking, has recently proven successful with a number of P-sheet breaking peptides. ^{3,4} The low-molecular-weight peptides, primarily fragments or analogs of the native Aβ, were shown to solubilize the Aβ fiber, supposedly by forming a non-covalent complex with the soluble conformation of Aβ. Such an activity of the β-sheet breaking peptides resulted not only in the in vitro solubilization of the fiber, but also in the in vivo reduction of plaques in mice.

We claim here that the effect of Aβ fiber solubilization, which may eventually prove to be an approach to the treatment of Alzheimer's disease, can be achieved by using small molecules of non-peptide nature. We describe a number of classes of compounds that, according to computer-aided design, are able to provide such a solubilization effect. We also describe the method for designing and testing such molecules for their in vitro and in vivo activity.

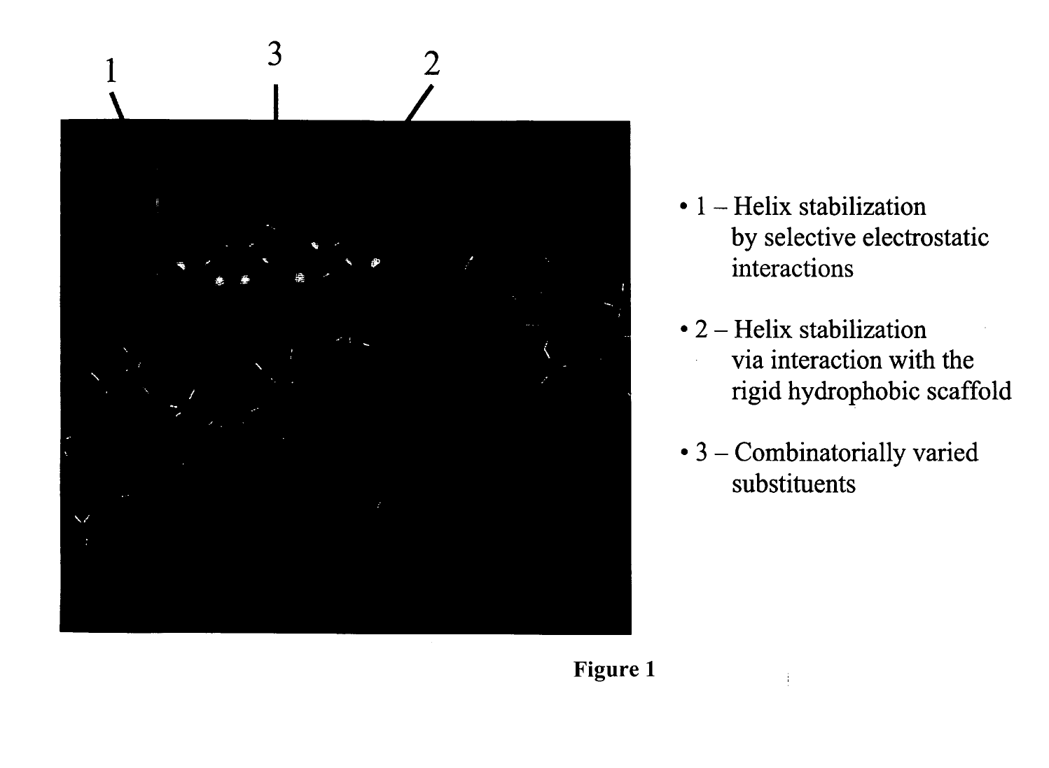

The small molecules described herein are generated from combinatorial libraries of compounds based on the scaffold structures 1-3 or similar compounds that can form non-covalent complexes with the soluble form of Aβ. As shown in FIG. 1, the binding of the compounds to Aβ is achieved via combination of electrostatic, hydrophobic, hydrogen-bonding and other kinds of non-covalent interactions with the Aβ α-helical form. The scaffold structures 1-3 have been designed with the aid of computer-assisted molecular modeling, taking into account the projected properties of the library components to facilitate their crossing the blood-brain barrier (BBB).^{5,6} The substituents R₁-R₃ and AA₁, AA₂ are varied within combinatorial libraries of the potential β-sheet breakers and the resulting component show different activity in the solubilization assays described below.

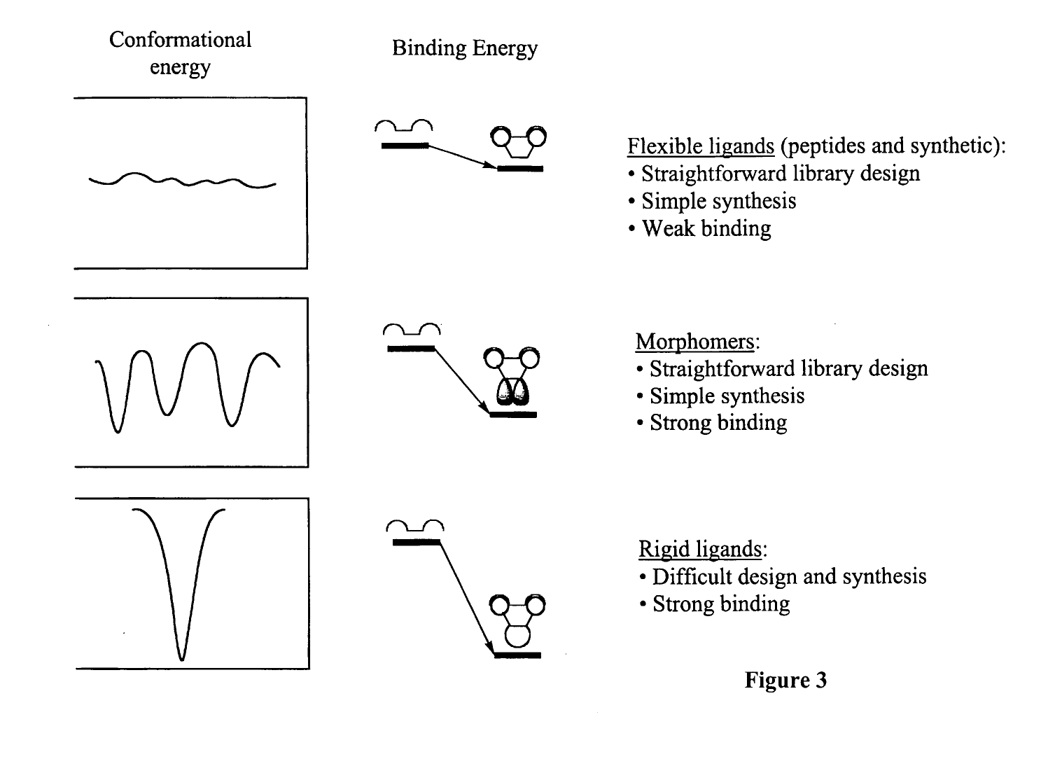

We also claim here a new concept in the design of the entities engaged in the molecular recognition, i.e. in formation of non-covalent complexes with biological targets, as depicted in pages 4-5.⁷ Such entities, defined as morphomers, include a number of bonds, around which the intramolecular rotation is slower than around average single bonds, such as e.g. sp³ carbon-carbon bond. Although the conformational change occurs in solution, it is sufficiently slow so that corresponding conformers can be observed, for example, as individual peaks in the NMR spectrum of the compounds. The examples of such conformationally restricted bonds are shown with arrows in structures 1-3. The different conformational states of these compounds, named morphomers, therefore form a sub-library of the main combinatorial library. Two properties of the morphomers are important for pharmaceutical applications: (i) binding energy to the target differs from one morphomer to the other; (ii) binding energy of a morphomer that has a better geometrical and functional fit to the target is higher than that of a similar compound with unrestricted conformations, as shown in FIG. 3.

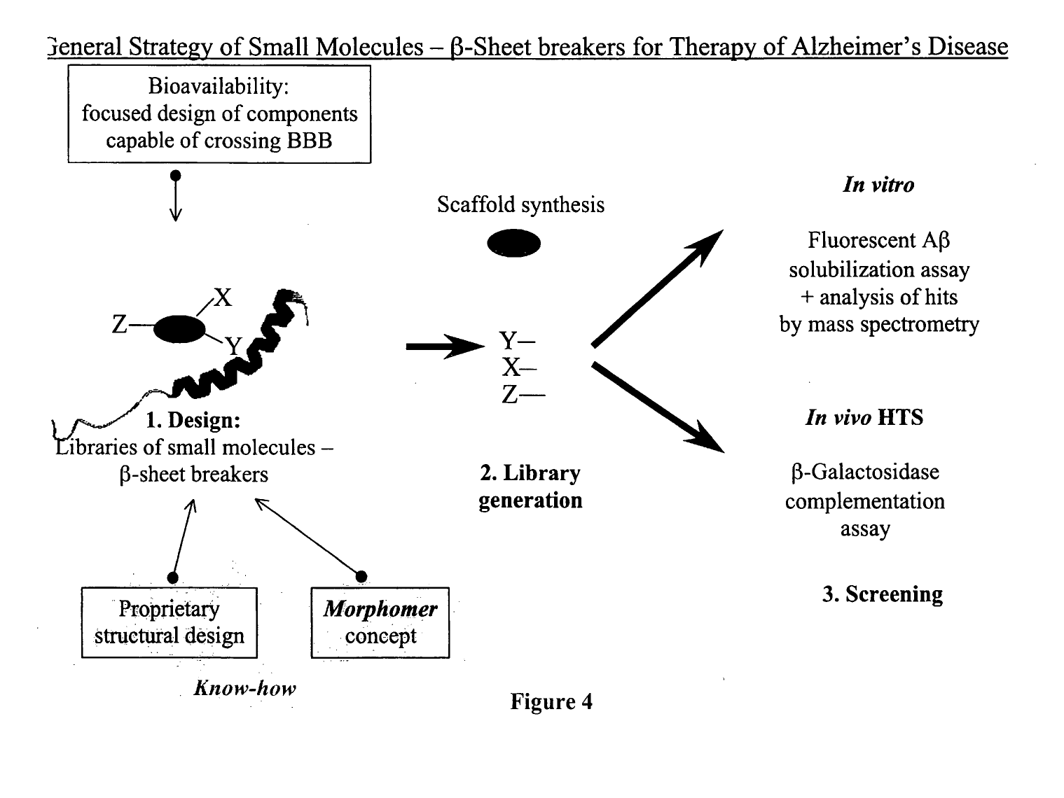

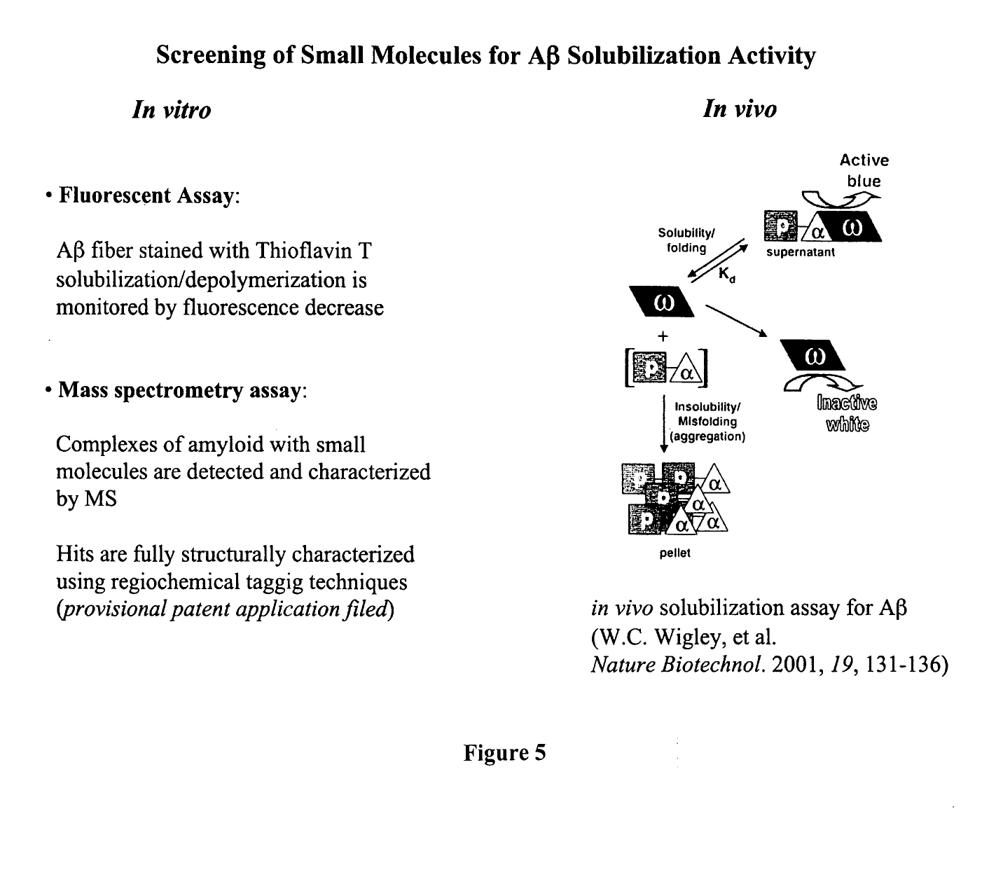

A general strategy for the discovery of small molecule- β-sheet breakers in shown in FIG. 4. It consists of computer-aided design of the molecular scaffolds, their synthesis, formation of the combinatorial libraries, and their screening in vitro and in vivo using known procedures. The in vitro assay is performed with either thioflavin T staining assay or by direct detection of Aβ complexes with library members by mass spectrometry. The in vivo assay has been recently published (ref. FIG. 5)

### REFERENCES

(1) Soto, C. Mol. Med. Today 1999, 5, 343-350.
(2) Jarvet, J.; Damberg, P.; Bodell, K.; Eriksson, L. E. G.; Graslund, A. J. Am. Chem. Soc. 2000, 122, 4261-4268.
β) Poduslo, J. F.; Curran, G. L.; Kumar, A.; Frangione, B.; Soto, C. J. Neurobiol 1999, 39, 371-382.
(4) Sigurdsson, E. M.; Permanne, B.; Soto, C.; Wisniewski, T.; Frangione, B. J.
Neuropathol. Exp. Neurol2000, 59, 11-17.
(5) Begley, D. J. Journal of Pharmacy & Pharmacology 1996, 48, 136-146.
(6) Cecchelli, R.; Dehouck, B.; Descamps, L.; Fenart, L.; Buee-Scherrer, V.; Duhem, C.; Lundquist, S.; Rentfel, M.; Torpier, G.; Dehouck, M. P. Adv. Drug Deliv. Rev 1999, 36, 165-178.
(7) Bunyapaiboonsri, T.; Lehn, J.-M. unpublished work.

1. A composition comprising small molecules for breaking of β-sheet conformation in amyloid protein.
